(19) United States
(12) Patent Application Publication
(10) Pub. No.: US 2026/0355010 A1
(43) Pub. Date: Nov. 12, 2026

(54) Portable Cold Therapy Sleeping Bag and Wearable Suit System

(71) Applicant: BHVD Labs, Inc., Chicago, IL (US)

(72) Inventor: Ira Ferencz, Chicago, IL (US)

(21) Appl. No.: 19/674,693

(22) Filed: May 12, 2026

Related U.S. Application Data
(60) Provisional application No. 63/804,069, filed on May 12, 2025.
(60) Provisional application No. 63/868,258, filed on Aug. 21, 2025.

(51) Int. Cl. A61F 7/10; A61F 7/00

(52) U.S. Cl. CPC A61F 7/106; A61F 2007/0001; A61F 2007/0095

(57) ABSTRACT

A cold containment system includes: a multi-layer bag including a middle phase change layer encapsulated between an outer insulative containment layer and an inner user contact layer; wherein, the full-body bag is further configured to open along its length to enable a user to enter and exit the bag when open and for the user’s lower body, torso, and arms to be entirely surrounded by the bag when closed; wherein the phase change layer includes a plurality of matrices of phase change material configured to be positioned along one or more of the lower body, torso, and arms of the user when the bag is closed around the user; and wherein, when starting from a temperature of 0 degrees Celsius, the bag delivers skin contact temperatures to a user in the bag along the user contact layer of between 0 to 10 degrees Celsius for at least 15 minutes.

### CROSS-REFERENCE TO PRIOR APPLICATIONS

The present application claims the benefit of priority to U.S. Provisional Patent Application No. 63/868,258, filed on Aug. 21, 2025, and claims the benefit of priority to U.S. Provisional Patent Application No. 63/804,069, filed on the entireties of which are incorporated herein by reference.

### BACKGROUND OF THE INVENTION

The present subject matter relates generally to systems and methods relating to cold containment systems and methods. More specifically, the present invention relates to cold containment system and methods utilizing full-body, self-contained, reusable, portable, cold containment systems employing passive and/or active cooling modules.

Cold therapy, or cryotherapy, is widely used for athletic recovery, stress reduction, and nervous system modulation. Conventional approaches to cold therapy include the use of ice packs, coolant sprays, ice massages, whirlpools, and ice baths. The conventional approaches typically suffer from being messy, space-intensive, and/or provide inconsistent or localized effects. For example, ice baths and chilled-water immersion require large volumes of water, active chillers, sanitation, and a dedicated space. Wearable passive cryotherapy wraps and powered cryotherapy modules generally treat localized regions and cannot deliver systemic nervous system effects.

An increasing number of users wish to experience the effects of cold therapy, but are limited by time commitment required by the typical messiness and location restrictions. There may be a benefit to taking a mid-day break from work or school to experience a five to ten minute, full-body, immersive cold therapy session, but managing the logistics can be difficult.

Accordingly, there is a need for a cold containment systems and methods, as described herein.

### BRIEF SUMMARY OF THE INVENTION

To meet the needs described above and others, the present disclosure provides systems and methods for a self-contained, reusable, transportable, cold containment system to provide full-body neurophysiological reset without requiring immersion in ice or water.

In a preferred embodiment, the systems and methods taught herein provide a multi-layer, full-body, wearable bag in which a plurality of cooling elements are distributed to provide uniform cooling to each of the torso, arms, and lower body of a user. In other examples, the cooling elements may be configured to direct cooling towards a subset of user body parts. In still other examples, the wearable bag and cooling elements may further provide cooling to the user’s neck and head, for example, by including a collar and a hood. In other examples, the cooling elements may be configured to provide less uniform and/or concentrated cooling. In some examples, user controls may be provided to direct the operation of active cooling elements. In other examples, the cooling elements may be passive cooling elements.

In a primary example, the cooling elements are thin and flexible phase change elements. For example, the phase change elements may utilize a sealed grid or molded pouch system containing a skin-safe, non-toxic hydrogel. In one embodiment, the hydrogel may include 60%-70% water, 10-20% glycerin or propylene glycol, and stabilizers such as sodium polyacrylate or silica gel. In another

In another example, the cooling elements may be formed using phase change material laminates including micro-encapsulated paraffin (e.g., C16 or C18) and/or a salt hydrate. Such laminates may be stacked in layers and sub-layers to provide the appropriate cooling and flexibility characteristics.

The pouches, laminates, or other encapsulation mechanisms may range in thickness depending on their location within the system, their thermal demands, and their need to be flexible. In some instances, the thickness of the pouches or other encapsulation mechanisms may range in thickness between 3.5mm and 12mm. In other instances, the thickness of the pouches or other encapsulation mechanisms may range in thickness between 0.3mm and 15mm.

The pouches, laminates, or other encapsulation mechanisms may be laminated, radio-frequency welded, ultrasonically bonded, adhesively sealed, or otherwise sealed to resist leakage under repeated freeze-thaw cycles.

In one embodiment, the full-body, wearable bag may be shaped as a single large sleeping bag style pouch. In other examples, the full-body, wearable bag may take the form of a snowsuit-like enclosure in which there are separate arm, leg, and torso portions. In such an embodiment, a matrix of pouches or other phase change element encapsulation mechanisms may be distributed throughout flexible garment panels with articulated joints to encourage better mobility.

In a preferred embodiment, the systems and methods taught herein provide a multi-layer, full-body, wearable bag including a cooling layer encapsulated between an outer insulating layer and an inner user contact layer. The three layers cooperate to form a shell of the wearable bag. In some embodiments, the term “outer layer” is used to describe the outermost surface of the wearable bag and the term “inner layer” is used to describe the innermost surface of the wearable bag. For example, an embodiment in which the cooling elements include phase change material in a molded pouch, the outward facing surface of the molded pouch may be the outer layer and the inward facing surface of the molded pouch may be the inner layer.

However, the multi-layer, full-body, wearable bag may alternatively be configured such that a phase change material is contained within a molded pouch and the molded pouch in contained within a fabric layer and the fabric layer within which the molded pouch is contained embodies a middle phase change layer which is then further encapsulated within a distinct outer layer and a distinct inner layer.

In some embodiments, the outer layer is an outer insulative containment layer formed from an insulated nylon, laminated polyester, or an aerogel composite material that insulates the inside of the bag from the outer environment to minimize thermal transfer through the outer layer of the bag. Similarly, the outer layer may include a thermally reflective material to minimize heat transfer through the layer.

In some embodiments, the inner layer is an inner user contact layer formed from a hypoallergenic, antimicrobial, moisture-wicking fabric such as bamboo viscose, polyester, or similar material.

Access into and out of the wearable bag may be facilitated by a closure mechanism using one or more of zippers (including one-way and two-way zippers), magnetic closures, hook-and-loop fasteners, ties, overlapping panels (e.g., elasticized panel openings), etc.

Although primarily described herein as a full-body wearable bag, the systems and methods taught herein may be embodied in bags or garments that are less than full-body wearable bags. For example, a bag or garment embodying the teachings provided herein may be configured to only cover a user’s lower body, or only the user’s lower body and torso.

In use, the multi-layer, full-body, wearable bag is configured to provide a full-body cold containment environment for a user for a therapeutically relevant duration of time. For example, a user may desire to be immersed in a temperature of approximately five degrees Celsius for a period of five to ten minutes. Because the multi-layer, full-body, wearable bag described herein can hold a low temperature for an extended period, a user may have flexibility in timing their use of the bag. For example, a user may store the bag folded in a residential freezer overnight. In other examples, the bag may reach operating temperature after being in a freezer for between four to eight hours. Once cooled, the user may then take the bag out of the freezer and transport it to a location in which the cold containment, i.e., cold immersion, activity will occur. The user may then conduct a ten-minute cold containment session before the bag loses its ability to hold a temperature of approximately five degrees Celsius. An additional insulated transportation bag may be used to contain the wearable bag and help to maintain its temperature during transportation.

In some embodiments, starting from a temperature of zero degrees Celsius, and without any active cooling, the wearable bag may hold a temperature of below 20 degrees Celsius for up to 45 minutes. In another example, starting from a temperature of zero degrees Celsius, and without any active cooling, the wearable bag may hold a temperature of below ten degrees Celsius for up to 45 minutes.

In one embodiment, a cold containment system includes: a multi-layer bag including a middle phase change layer encapsulated between an outer insulative containment layer and an inner user contact layer; wherein, the full-body bag is further configured to open along a length of the bag to enable a user to enter and exit the bag when open and for the user’s lower body, torso, and arms to be entirely surrounded by the bag when closed; wherein the phase change layer includes a plurality of matrices of phase change material configured to be positioned along one or more of the lower body, torso, and arms of the user when the bag is closed around the user; and wherein, when starting from a temperature of 0 degrees Celsius, the bag delivers skin contact temperatures to a user in the bag along the user contact layer of between 0 to 10 degrees Celsius for at least 15 minutes.

The cold containment system may include a plurality of temperature monitoring sensors located along the user contact layer. The temperature monitoring sensors may communicate temperature data through an associated display. The display may be associated with a user device with which the temperature monitoring sensors communicate wirelessly. In such embodiments, a user may monitor the internal temperature of the bag over its use to help to maintain an appropriate balance of temperature and duration for a cold therapy session. The temperature monitoring sensors may be located throughout the wearable bag or concentrated in specific zones. One or more temperatures may be reported or displayed to the user indicating, for example, a temperature for each of a torso, a left arm, a right arm, a left leg, and a right leg. Reporting the temperature by zones may enable a user not only to better monitor a given cold therapy session, but also to better monitor the performance of the wearable bag over time.

The multi-layer bag may be foldable. The multi-layer bag may be foldable to a size of less than 20 inches by 20 inches by 10 inches. Obviously, the construction of the bag and its multiple layers will affect its ability to be folded. In a preferred embodiment, the multi-layer bag may be folded small enough to fit inside of a typical residential freezer to make the system more user friendly to prepare for use. For example, the multi-layer bag may be foldable to a size of less than 15 inches by 15 inches by 5 inches. Obviously, the foldable size of the multi-layer bag may vary depending on the materials and construction used in the multi-layer bag.

The cold containment system may include an insulated transportation bag within which the multi-layer bag can be contained to maintain its temperature during transportation. Such insulated transportation bag may include a sealable insulated space within which a folded multi-layer bag may be stored and transported, thereby prolonging the time the multi-layer bag maintains its cold temperature.

The outer insulative containment layer may include an insulative material including one of insulated nylon, laminated polyester, or aerogel composites.

As an element of the middle phase change layer, the phase change material itself may be between 0.3 mm to 15.0 mm thick. The phase change material may be contained within cells. The cells may be arranged in a pattern, such as a hexagonal grid. Alternatively, the cells may be arranged in a triangular, wave, tessellated, or irregular pattern. The cells may be sealed to resist leakage over repeated freeze-thaw cycles by one of lamination, radio-frequency welding, ultrasonic bonding, or adhesion.

The inner user contact layer may include a hypoallergenic, antimicrobial, moisture wicking fabric. For example, the hypoallergenic, antimicrobial, moisture wicking fabric may be bamboo viscose or polyester. The inner contact layer may be formed from any material appropriate for a user contact surface.

The full-body bag may include a collar configured to surround a portion of the user’s neck, wherein the collar is formed from the middle phase change layer encapsulated between the outer insulative containment layer and the inner user contact layer. Similarly, the full-body bag may include a hood, wherein the hood is formed from the middle phase change layer encapsulated between the outer insulative containment layer and the inner user contact layer.

The full-body bag may open and close using a closure mechanism. The closure mechanism may include one of a zipper, a magnetic closure, a tie, and hook and loop fasteners.

The cold containment system may include active cooling augmentation provided by one of a fan, a refrigeration element, and a thermoelectric module. An active cold containment system may be AC or battery powered. The use of active cooling elements within a cold containment system may enable a user greater control over temperature, both the specific choice of temperature and the ability to hold a specific temperature, or control a specific temperature profile, over time.

In another embodiment, a cold containment system includes: a foldable multi-layer bag including a middle phase change layer encapsulated between an outer insulative containment layer and an inner user contact layer; wherein, the full-body bag is further configured to open along a length of the bag to enable a user to enter and exit the bag when open and for the user’s lower body, torso, and arms to be entirely surrounded by the bag when closed and the user’s neck to be at least partially surrounded by the bag when closed, wherein the collar is formed from the middle phase change layer encapsulated between the outer insulative containment layer and the inner user contact layer, wherein the full-body bag opens and closes using a zippered closure mechanism; wherein the phase change layer includes a plurality of matrices of phase change material configured to be positioned along one or more of the lower body, torso, and arms of the user when the bag is closed around the user, wherein the phase change material is contained within cells are arranged in a hexagonal, triangular, wave, tessellated, or irregular pattern and sealed to resist leakage over repeated freeze-thaw cycles; and wherein, when starting from a temperature of 0 degrees Celsius, the bag delivers skin contact temperatures to a user in the bag along the user contact layer of between 0 to 10 degrees Celsius for at least 15 minutes.

An object of the subject matter taught herein is to provide a self-contained, reusable, transportable, cold containment system to provide full-body neurophysiological reset without requiring immersion in ice or water.

Another object of the subject matter taught herein is to provide a full-body, dry, cold containment system.

An additional object of the subject matter taught herein is to deliver up to 45 minutes of full-body, controlled, skin-contact temperatures between 0 degrees Celsius and 20 degrees Celsius.

A further object of the subject matter taught herein is to provide a full-body, foldable, freezer compatible cold containment system.

An advantage of the subject matter taught herein is it provides a portable, full-body cold containment system.

Another advantage of the subject matter taught herein is it eliminates the use of water and ice in a full-body cold containment system.

A further advantage of the subject matter taught herein is it is reusable across multiple freeze-thaw cycles.

Another advantage of the subject matter taught herein is to provide a cold containment system designed for direct skin-safe contact by the user.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 is an isometric view of a body suit shaped, multi-layer, full-body, wearable bag embodying a cold containment system.

FIG. 2 is an isometric view of a sleeping bag shaped, multi-layer, full-body, wearable bag embodying a cold containment system.

FIG. 3 is an isometric view of a sleeping bag shaped, multi-layer, full-body, wearable bag embodying a cold containment system including a collar portion.

FIG. 4 is a cross-sectional view of the multi-layer construction of the cold containment system shown in each of FIGS. 1-3.

FIG. 5 is an isometric view of the containment system shown in FIG. 3 in a folded configuration.

FIG. 6 is an isometric view of an insulated transportation bag for storing and transporting the containment system shown in FIG. 3 in a folded configuration.

FIG. 7 is the containment system shown in FIG. 2, illustrating a hexagonal configuration of cooling elements.

FIG. 8 is the containment system shown in FIG. 2, illustrating a plurality of sensors that remotely communicate temperature information to a display.

FIG. 9 is the containment system shown in FIG. 2, illustrating a plurality of active cooling elements.

### DETAILED DESCRIPTION OF THE INVENTION

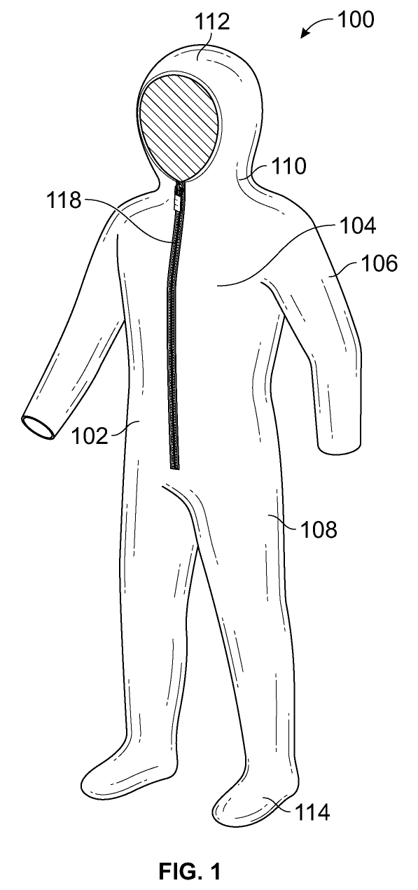

FIG. 1 shows an example of a cold containment system 100 embodied in a multi-layer, full-body, wearable bag 102 in the form of a full-body suit in which a plurality of cooling elements located within the wearable bag 102 are distributed to provide uniform cooling to each of the torso, arms, and lower body of a user via a torso portion 104, two arms portions 106, and two lower body portions 108, respectively. As further shown in FIG. 1, the wearable bag 102 and cooling elements may further provide cooling to the user’s neck and head by including a collar 110 and a hood 112 and may provide cooling to the user’s feet by including foot portions 114.

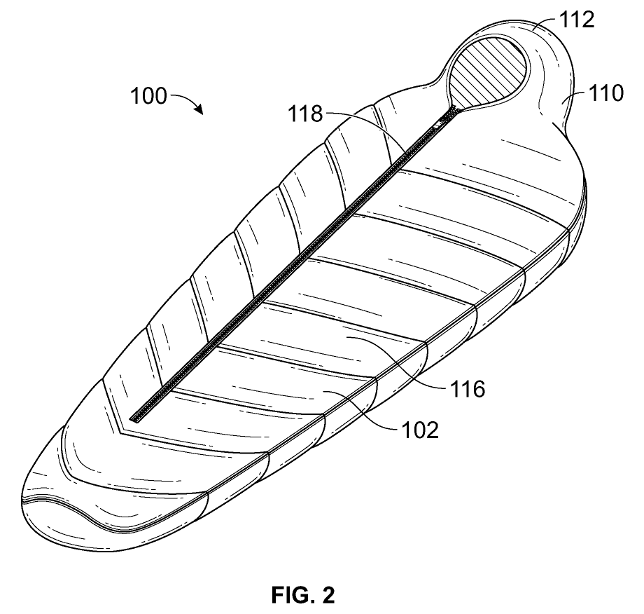

FIG. 2 shows and example of a cold containment system 100 embodied in a multi-layer, full-body, wearable bag 102 in the form of a hooded sleeping bag in which a plurality of cooling elements located within the wearable bag 102 are distributed to provide uniform cooling to each of the torso, arms, hands, and legs, and feet of a user via a full-body portion 116. As further shown in FIG. 1, the wearable bag 102 and cooling elements may further provide cooling to the user’s neck and head by including a collar 110 and a hood 112.

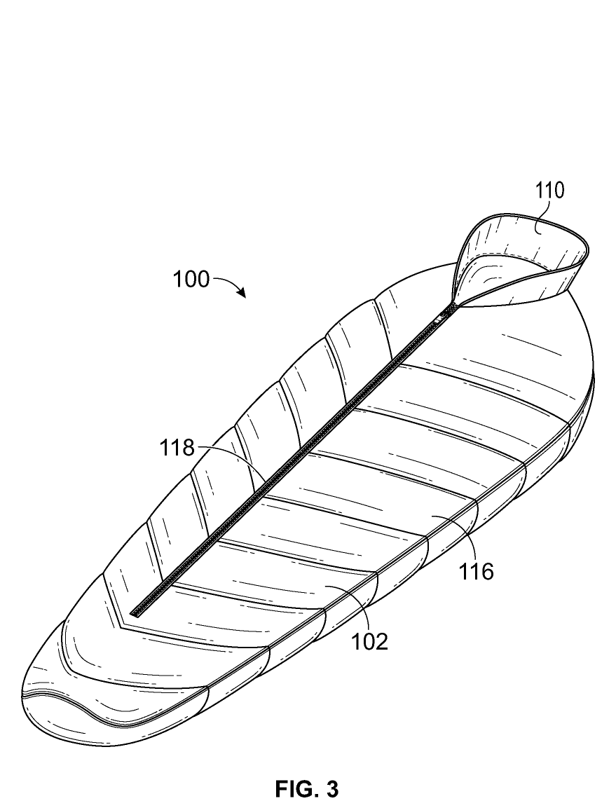

FIG. 3 shows and example of a cold containment system 100 embodied in a multi-layer, full-body, wearable bag 102 in the form of a collared sleeping bag in which a plurality of cooling elements located within the wearable bag 102 are distributed to provide uniform cooling to each of the torso, arms, hands, and legs, and feet of a user via a full-body portion 116. As further shown in FIG. 1, the wearable bag 102 and cooling elements may further provide cooling to the user’s neck by including a collar.

In each of the example of the cold containment system 100 shown in FIGS. 1-3, a closure mechanism 118 is provided to facilitate user access into and out of the wearable bag 102. The closure mechanism 118 shown in FIGS. 1-3 is a zipper. However, the closure mechanism 118 may be one or more of zippers (including one-way and two-way zippers), magnetic closures, hook-and-loop fasteners, ties, overlapping panels (e.g., elasticized panel openings through which the user simply climbs in and out), etc.

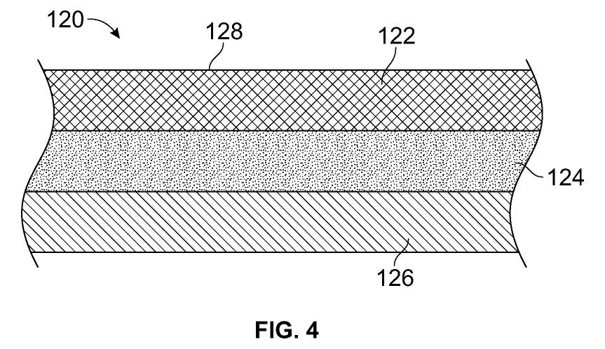

In the examples shown in FIGS. 1-3, the wearable bag 102 includes a three-layer fabric structure 120, including an outer layer 122, a middle layer 124, and an inner layer 126. A cross-section of the three-layer fabric structure 120 is shown in FIG. 4. As shown in FIG. 4, the middle layer 124 is encapsulated between the outer layer 122 and the inner layer 126.

In these examples, the middle layer 124 comprises a phase change material that is positioned along the lower body, torso, and arms of the user when the wearable bag 102 is closed around the user. As shown in the example provided in FIG. 4, the entire middle layer 124 may be comprised of a phase change material. In alternative embodiments, the middle layer 124 may include a matrix of molded pouches or similar structures in which a phase change material is contained. In this example, the matrix of molded pouches forms the middle layer 124 which is encapsulated between the outer layer 122 and the inner layer 126.

In examples in which the entire middle layer 124 is formed from phase change material, the cooling effects may be applied consistently throughout the entirety of the wearable bag 102. In instances in which the middle layer 124 includes distinct matrixes of phase change material, the cooling effects may be specifically targeted toward the user’s body parts.

The outer layer 122 shown in FIG. 4 is an insulative containment layer formed from an insulated nylon, laminated polyester, or an aerogel composite material that insulates the inside of the wearable bag 102 from the outer environment to minimize thermal transfer through the outer layer 122 of the bag. The outer layer 122 shown in FIG. 4 may include a thermally reflective material 128 along its outermost surface to minimize heat transfer through the outer layer 122.

The inner layer 126 shown in FIG. 4 is a user contact layer formed from a hypoallergenic, antimicrobial, moisture-wicking fabric such as bamboo viscose, polyester, or similar material.

Each of the layers of the three-layer fabric structure 120 shown in FIG. 4 may be made of varying thickness. For example, it is contemplated that the middle layer 124, which includes the phase change material, may be between 0.3 mm to 15.0 mm thick. In some embodiments in which there are portions of the wearable bag 120 in which cooling elements are distributed along specific areas and not others, the three-layer fabric structure 120 may include portions in which there is a middle layer 124 sandwiched between the outer layer 122 and the inner layer 126 and portions in which the outer layer 122 and the inner layer 126 adjoin.

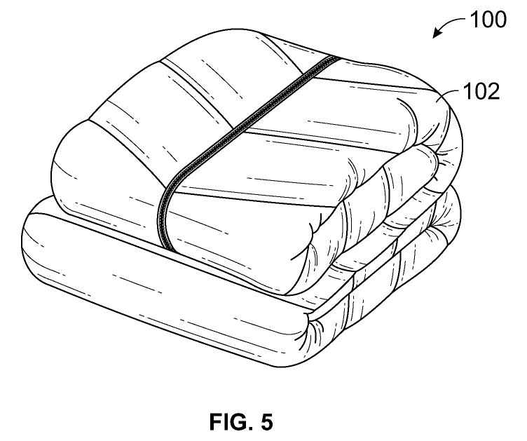

FIG. 5 shows the embodiment of the cold containment system 100 shown in FIG. 3 in a folded configuration. As shown, the wearable bag 102 may be foldable. Depending on the configuration of the wearable bag 102 and the thickness and flexibility of the various layers and cooling elements, the wearable bag 102 may be foldable to various sizes. In a preferred embodiment, the wearable bag 102 may be folded to a size of less than 20 inches by 20 inches by 10 inches and may be folded small enough to fit inside of a typical residential freezer to make the cold containment system 100 more user friendly to prepare for use in the typical user’s daily routine. In some examples, the multi-layer bag may be foldable to a size of less than 15 inches by 15 inches by 5 inches.

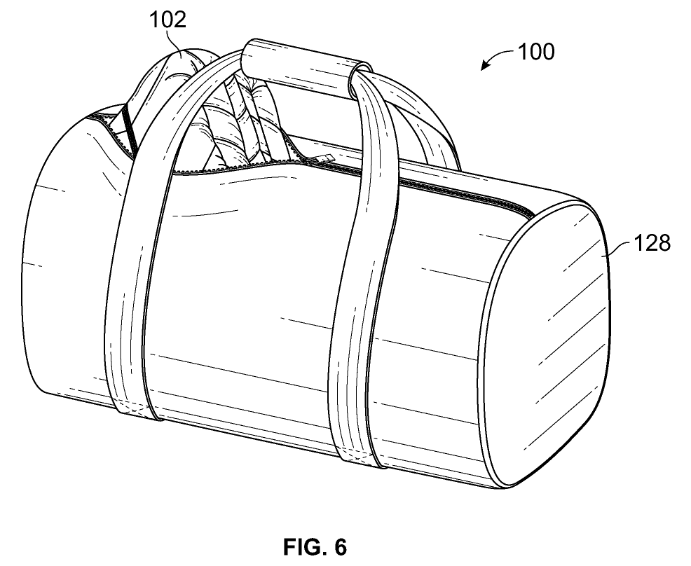

As shown in FIG. 6, the cold containment system 100 may include an insulated transportation bag 128 within which the folded wearable bag 102 can be contained to maintain its temperature during transportation. Such insulated transportation bag 128 may include a sealable insulated space within which the wearable bag 102 may be stored and transported, thereby prolonging the time the wearable bag 102 maintains its cold temperature. The insulated transportation bag 128 may be constructed as any standard insulated bag, including, for example, a durable outer shell made from polyester or nylon, a foam-based insulation core made from expanded polyethylene or polyurethane foam, and an inner liner made from polyethylene vinyl acetate, aluminum foil, or similar.

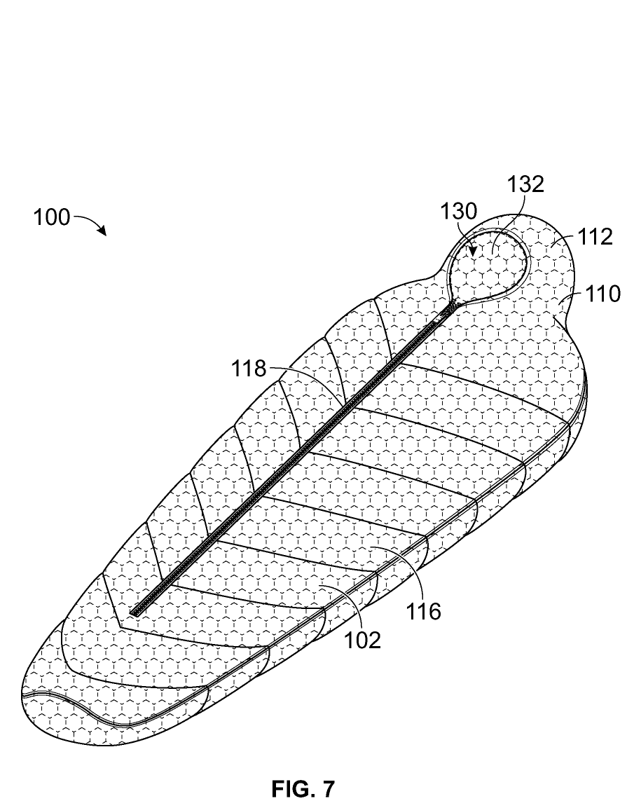

FIG. 7 shows the cold containment system 100 from FIG. 2, further illustrating a hexagonal matrix configuration of cooling elements 130. In the example shown in FIG. 7, the hexagonal matrix configuration of cooling elements 130 includes a plurality of hexagonal-shaped molded pouches 132 filled with phase change material. In this example, the hexagonal matrix configuration of cooling elements 130 spans the entire middle layer 124 of the wearable bag 102. However, it is contemplated that the pattern of cooling elements may be various shapes and sizes and may be limited to correspond to only specific user body parts or may be continuously and uniformly distributed throughout the wearable bag 102.

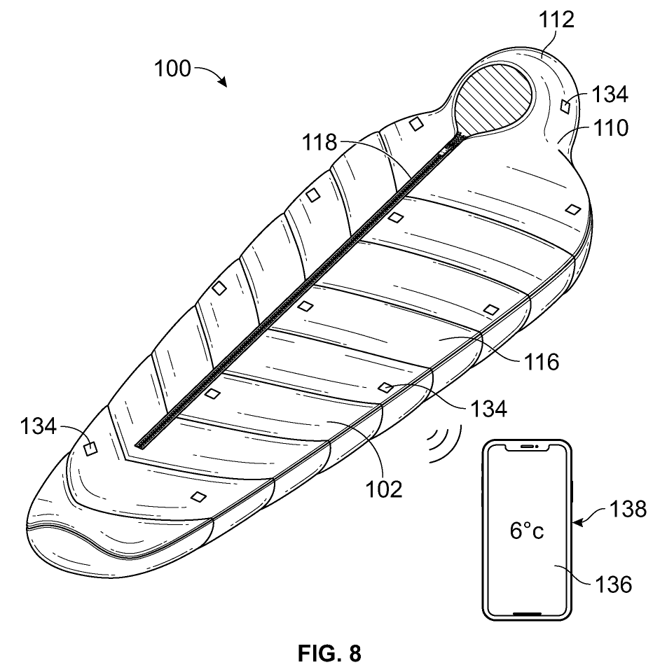

FIG. 8 is the containment system shown in FIG. 2, illustrating a plurality of sensors 134 that remotely communicate temperature information to an associated display 136 of a user device 138. In this example, the plurality of sensors 134 read the temperature at various positions within the wearable bag 102. In the example shown, the display 136 shows the average skin contact temperature in the wearable bag 102. In other examples, the display may provide a temperature for each specific sensor 134. The display may further provide additional information such as duration, rate of temperature change, etc.

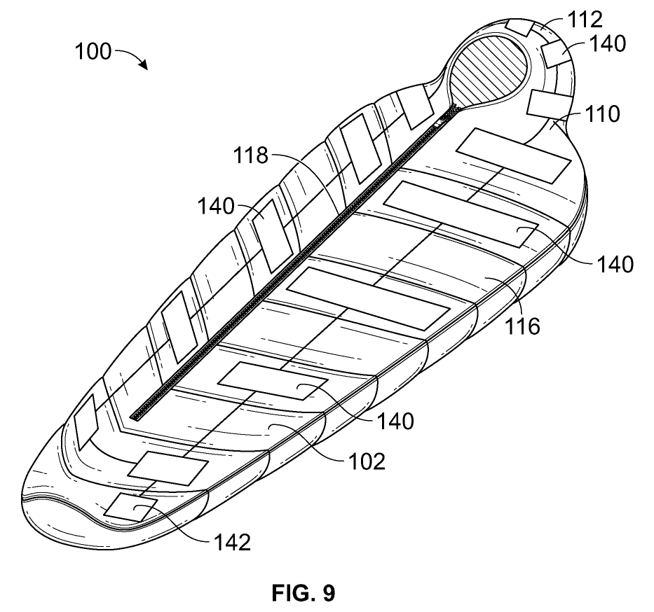

FIG. 9 is the containment system shown in FIG. 2, illustrating a plurality of active cooling elements 140 distributed throughout the wearable bag 102. In the example shown in FIG. 9, the active cooling elements 140 are thermoelectric cooling modules 140 powered by an integrated battery 142 that transfer heat from the inner layer 126 through the middle layer 124 (e.g., the active cooling elements 140 themselves) out through the outer layer 122. In such embodiments, the middle layer 124 (e.g., the active cooling elements 140 themselves) may be exposed to the ambient environment though the outer layer 122 to enable more efficient heat transfer through the cold containment system 100. In other words, the middle layer 124 may be vented such that the outer layer 122 doesn’t trap the heat transfer within the wearable bag 102. While shown as battery powered, in other embodiments, the active cooling elements 140 may be powered in any manner including one or more of an integrated power source (e.g., battery), a portable power source (e.g. generator), or a connection to mains electricity (e.g., a power supply connected to a utility grid).

For purposes of clarity and to reduce visual clutter, the plurality of sensors 134 shown in FIG. 8 and the active cooling elements 140 shown in FIG. 9 are shown in separate figures, but it is contemplated that both the plurality of sensors 134 and the active cooling elements 140 may be included in a single embodiment of the wearable bag 102. Similarly, any of the features and functions illustrated with respect to any one of FIGS. 1-9 may be used in combination with any one or more of the other features and functions illustrated with respect to any one of FIGS. 1-9.

It should be noted that various changes and modifications to the presently preferred embodiments described herein will be apparent to those skilled in the art. Such changes and modifications may be made without departing from the spirit and scope of the present invention and without diminishing its attendant advantages.

I claim:

1. A cold containment system, comprising:
a multi-layer bag including a middle phase change layer encapsulated between an outer insulative containment layer and an inner user contact layer;
wherein, the full-body bag is further configured to open along a length of the bag to enable a user to enter and exit the bag when open and for the user’s lower body, torso, and arms to be entirely surrounded by the bag when closed;
wherein the phase change layer includes a plurality of matrices of phase change material configured to be positioned along one or more of the lower body, torso, and arms of the user when the bag is closed around the user; and
wherein, when starting from a temperature of 0 degrees Celsius, the bag delivers skin contact temperatures to a user in the bag along the user contact layer of between 0 to 10 degrees Celsius for at least 15 minutes.

2. The cold containment system of claim 1, further including a plurality of temperature monitoring sensors located along the user contact layer.

3. The cold containment system of claim 2, wherein the temperature monitoring sensors communicate temperature data through an associated display.

4. The cold containment system of claim 3, wherein the display is associated with a user device with which the temperature monitoring sensors communicate wirelessly.

5. The cold containment system of claim 1, wherein the multi-layer bag is foldable.

6. The cold containment system of claim 5, wherein the multi-layer bag is foldable to a size of less than 20 inches by 20 inches by 10 inches.

7. The cold containment system of claim 1, further comprising an insulated transportation bag within which the multi-layer bag can be contained to maintain its temperature during transportation.

8. The cold containment system of claim 1, wherein the outer insulative containment layer includes an insulative material including one of insulated nylon, laminated polyester, or aerogel composites.

9. The cold containment system of claim 1, wherein the phase change material is between 0.3 mm to 15.0 mm thick.

10. The cold containment system of claim 1, wherein the phase change material is contained within cells.

11. The cold containment system of claim 10, wherein the cells are arranged in a hexagonal grid.

12. The cold containment system of claim 10, wherein the cells are arranged in a hexagonal, triangular, wave, tessellated, or irregular pattern.

13. The cold containment system of claim 10, wherein the cells are sealed to resist leakage over repeated freeze-thaw cycles by one of lamination, radio-frequency welding, ultrasonic bonding, or an adhesive.

14. The cold containment system of claim 1, wherein the inner user contact layer includes a hypoallergenic, antimicrobial, moisture wicking fabric.

15. The cold containment system of claim 14, wherein the hypoallergenic, antimicrobial, moisture wicking fabric is bamboo viscose or polyester.

16. The cold containment system of claim 1, wherein the full-body bag includes a collar configured to surround a portion of the user’s neck, wherein the collar is formed from the middle phase change layer encapsulated between the outer insulative containment layer and the inner user contact layer.

17. The cold containment system of claim 1, wherein the full-body bag opens and closes using a closure mechanism.

18. The cold containment system of claim 17, wherein the closure mechanism includes one of a zipper, a magnetic closure, and hook and loop fasteners.

19. The cold containment system of claim 1, further comprising active cooling augmentation provided by one of a fan, a refrigeration element, and a thermoelectric module.

20. A cold containment system, comprising:
a foldable multi-layer bag including a middle phase change layer encapsulated between an outer insulative containment layer and an inner user contact layer;
wherein, the full-body bag is further configured to open along a length of the bag to enable a user to enter and exit the bag when open and for the user’s lower body, torso, and arms to be entirely surrounded by the bag when closed and the user’s neck to be at least partially surrounded by the bag when closed, wherein the collar is formed from the middle phase change layer encapsulated between the outer insulative containment layer and the inner user contact layer, wherein the full-body bag opens and closes using a zippered closure mechanism;
wherein the phase change layer includes a plurality of matrices of phase change material configured to be positioned along one or more of the lower body, torso, and arms of the user when the bag is closed around the user, wherein the phase change material is contained within cells are arranged in a hexagonal, triangular, wave, tessellated, or irregular pattern and sealed to resist leakage over repeated freeze-thaw cycles; and
wherein, when starting from a temperature of 0 degrees Celsius, the bag delivers skin contact temperatures to a user in the bag along the user contact layer of between 0 to 10 degrees Celsius for at least 15 minutes.
